(19) United States
(12) Patent Application Publication
(10) Pub. No.: US 2025/0029680 A1
(43) Pub. Date: Jan. 23, 2025

(54) DRUGGABILITY SCORE-BASED RANKING AND LIGAND TYPE CLASSIFICATION OF PROTEIN-LIGAND BINDING SITES

(71) Applicant: Ainnocence INC, MIAMI, FL (US)

(72) Inventors: Orhun Vural, Hoover, AL (US); Lurong Pan, Vestavia Hill, AL (US)

(21) Appl. No.: 18/355,308

(22) Filed: Jul. 19, 2023

(51) Int. Cl. G16B 40/00; G16B 15/20; G16B 35/10; G16B 35/20

(52) U.S. Cl. CPC G16B 40/00; G16B 15/20; G16B 35/10; G16B 35/20

(57) ABSTRACT

The present invention enables the prediction of both the ligand type for protein-ligand binding sites and their druggability scores. The invention provides a computational method to investigate the ligand type and druggability of protein-ligand binding sites. The invention has two distinct training sets for two different prediction methods. The method leverages attention-based deep learning models for both ligand type and druggability prediction tasks. Deep learning models incorporate both channel-based and spatial-based attention mechanisms. The training phase focuses on the coordinates of known ligand binding sites to enhance the accuracy of druggability prediction. The method also provides the capability to update the training set with new data, thereby ensuring continued improvement in the prediction performance. The invention also details a computer device with a memory and processor, storing a computer program to implement the method. The device processes input in the PDB format, performs the necessary cleaning steps, and utilizes the trained models to predict ligand binding site's types and their respective druggability scores. The results are integrated into a table representation and presented to the user, offering an efficient way to understand and apply the predictions.

### TECHNICAL FIELD OF THE INVENTION

The invention pertains to the domain of protein-ligand binding site analysis and is particularly relevant in the classification of protein-ligand binding sites based on ligand types as well as the computation of binding sites' druggability scores.

### BACKGROUND OF THE INVENTION

Drug discovery has long been a time-consuming and costly process. The scoring of protein-ligand binding sites in terms of druggability and the grouping of pockets by the types of ligands are important parts of drug design studies that involve many different and complex steps. It is important to determine the druggability score accurately because it guides drug discovery, optimizes resource allocation, aids rational drug design, and enables the repurposing of existing drugs, ultimately leading to more effective therapies for various diseases. Likewise, having knowledge about the most likely type of ligand to bind to the protein's binding sites is important in drug design. This knowledge can make it possible to create individualized pharmaceuticals that can control the activity of the protein.

The currently used approaches for calculating the druggability of protein-ligand binding sites do not produce accurate findings at a satisfactory level. Apart from ranking druggability scores, our invention also predicts the types of ligands that are likely to bind to the protein-ligand binding sites. The specificity of a protein's binding site refers to its ability to selectively identify and form bonds with specific ligands or molecules. Laboratory-based approaches examine the binding preferences of protein-ligand binding sites for specific ligand types. Instead of relying on experimental methods, we propose a computational approach within the structure-based drug design (SBDD) pipeline. The name of our invention is Druggability Score-Based Ranking and Ligand Type Classification of Protein-Ligand Binding Sites (RCLigand). When the RCLigand program is executed, the output that is shown in Table 1 is generated. Our invention takes protein files in protein data bank (PDB) format as input and generates outputs specific to each protein, as shown in Table 1. The PDB file format is used to store information about the atoms that make up a macromolecule, their positions in space, and the bonds between them. The techniques we used while creating Table 1 and the dataset we created by labeling make our study unique. The process of generating the dataset is illustrated in FIG. 1. The output of running the RCLigand invention is displayed in Table 1. FIG. 2 provides a comprehensive depiction of the step-by-step process for creating Table 1. The training process of the deep learning models utilized in RCLigand is illustrated in FIG. 3.

### SUMMARY OF THE INVENTION

The invention has two important parts, one of which is the scoring of protein-ligand binding sites according to their druggability probability and their ranking, and the other is the prediction of these protein-ligand binding sites according to their ligand types. To perform these two steps, two distinct datasets are required. The first dataset is for the deep learning model that will be trained to predict the druggability score The second dataset is for another deep learning model that will be trained to predict the ligand types of the protein-ligand binding sites. scPDB, COACH420, HOLO4K, and PDBbind are the primary databases used to create the two distinct datasets. There is a need for a dataset that should be prepared meticulously and labeled according to the ligand types for the proposed model. Similarly, to accurately predict the druggability score, it is necessary to meticulously prepare a labeled another dataset.

The preparation of the dataset to be used to predict the ligand types of the protein-ligand binding sites is shown in FIG. 1. During dataset preparation, Protein Data Bank (PDB) extension files were used to represent the proteins, and Structure Data File (SDF) extension files were used to represent the ligands that bind to these proteins. To prepare our dataset, we require two types of files. First, PDB files that contain the structure of the protein, and second, SDF files that contain the structure of the ligand known to bind to that protein. Any dataset that includes these two file types is sufficient for our dataset preparation process. For instance, the PDBbind and ScPDB datasets each include PDB files as well as relevant SDF for each protein. These datasets are used for studying protein-ligand interactions as well as developing and evaluating algorithms for predicting binding affinity within the fields of computational biology, chemistry, and so on. In our study, we downloaded Crystallographic Information File (CIF) format files for each protein in our dataset. From these files, we extracted information about the type of ligands that bind to the protein. Each protein is put into one of five groups based on the type of ligand. Protein-ligand binding sites were classified into five distinct groups, including antagonist, agonist, activator, inhibitor, and other types based on their response. If protein-ligand binding sites do not interact with any antagonist, agonist, activator, or inhibitor ligand types, they are labeled as ‘others’.

After preparing the dataset for training and testing, we can begin the development of the RCLigand model. FIG. 2 provides a detailed step-by-step illustration of how the RCLigand software predicts with a PDB input. FIG. 3 shows the training steps of deep learning models to be used for RCLigand software. The dataset obtained from FIG. 1 serves as the input data for step 301 in FIG. 3. As mentioned earlier, the invention comprises two main stages: calculating the druggability score of protein-ligand binding sites and predicting the ligand type for each protein-ligand binding site. The steps between 301 and 305 in FIG. 3 are the same for both ranking binding sites based on their druggability score and classifying protein-ligand binding sites based on the type of ligand. Steps 308, 309, 310 and 312 in FIG. 3 are training steps for predicting the protein-ligand binding sites' druggability score. The trained model obtained from these steps is utilized in step 205 of FIG. 2. Likewise, steps 306, 307, 310 and 311 in FIG. 3 are training steps for predicting the protein-ligand binding sites' ligand type. The trained model obtained from these steps is used to predict the ligand type in step 205 in FIG. 2. Consequently, in FIG. 2, the PDB input is obtained from the end user in step 201, followed by ligand type prediction in step 207, druggability score prediction in step 206, and ultimately, the combination of these two results in step 208, yielding Table 1.

### BRIEF DESCRIPTION OF DRAWINGS

In order to illustrate the technical solutions of the embodiments of the present application more clearly, the following briefly introduces the drawings that are used in the embodiments.

FIG. 1 is a schematic flow chart of the process of obtaining the categorized protein dataset based on ligand type;

FIG. 2 is a schematic flowchart illustrating the prediction process of Druggability Score-Based Ranking and Ligand Type Classification of Protein-Ligand Binding Sites (RCLigand), which utilizes input from the end user;

FIG. 3 is a schematic flowchart that illustrates the training process of two distinct deep learning models within the Druggability Score-Based Ranking and Ligand Type Classification of Protein-Ligand Binding Sites (RCLigand); and

Table 1 displays the ultimate outcome.

### DETAILED DESCRIPTION

The embodiments of that present disclosure are described in detail below with reference to the drawings. Obviously, the described embodiments are only a part of the embodiments of the present disclosure, but not all of the embodiments. The current disclosure may be implemented or applied by alternative embodiments, and the present specification may be amended or altered from various aspects and applications without straying from the spirit of the present application.

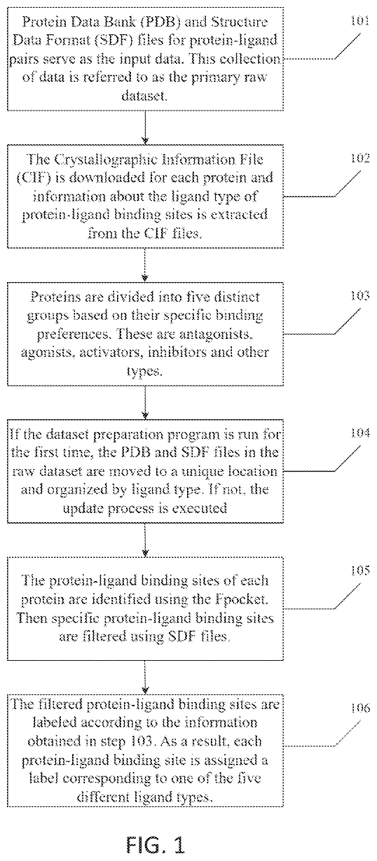

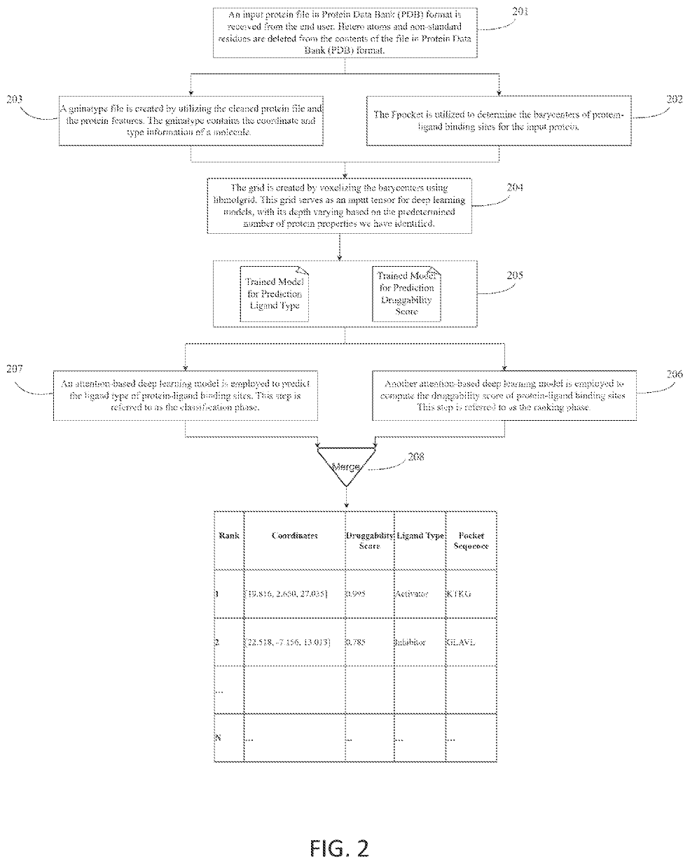

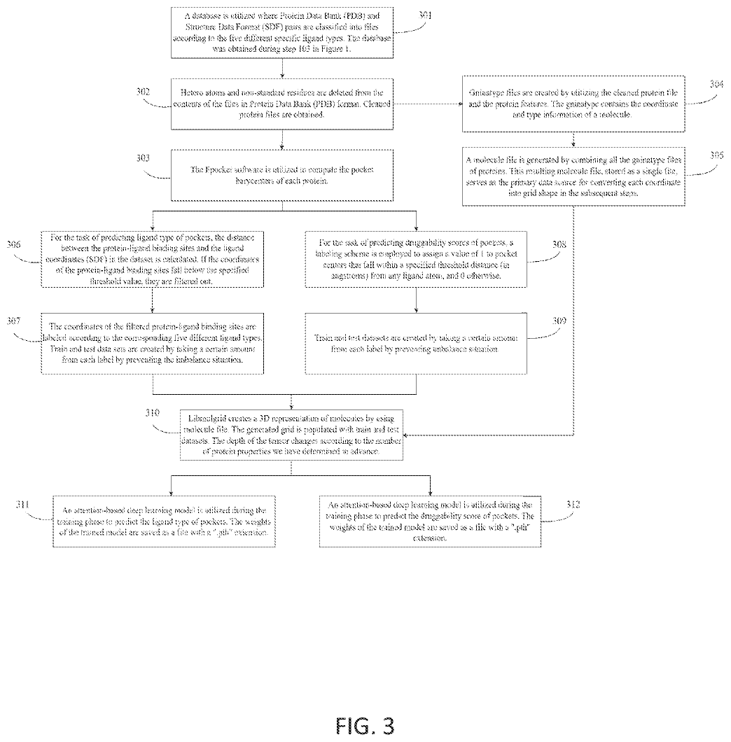

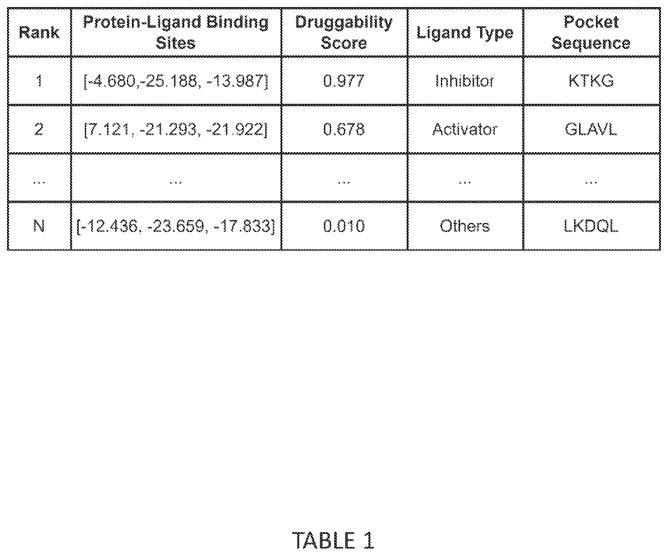

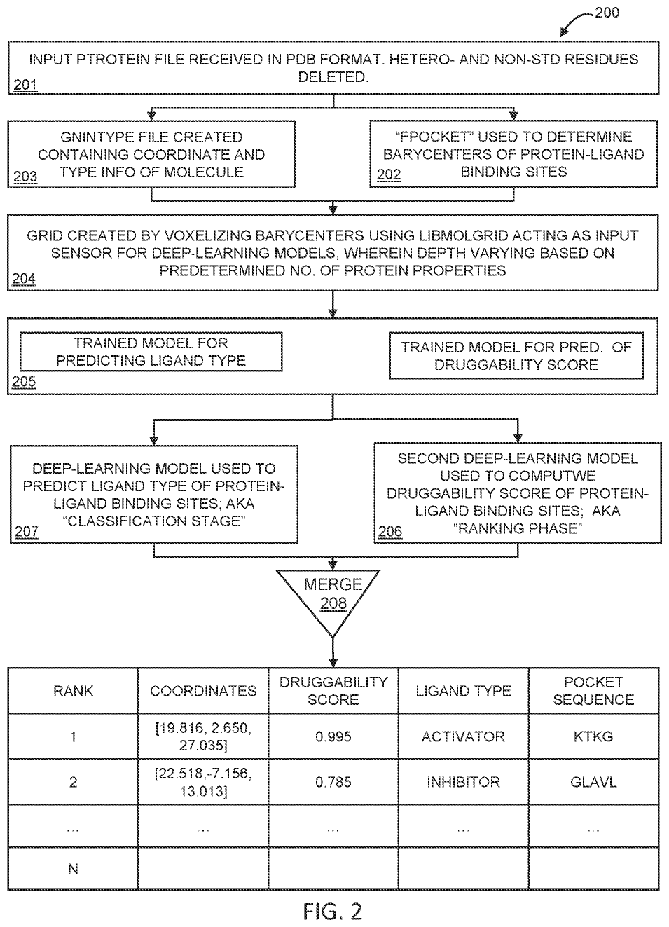

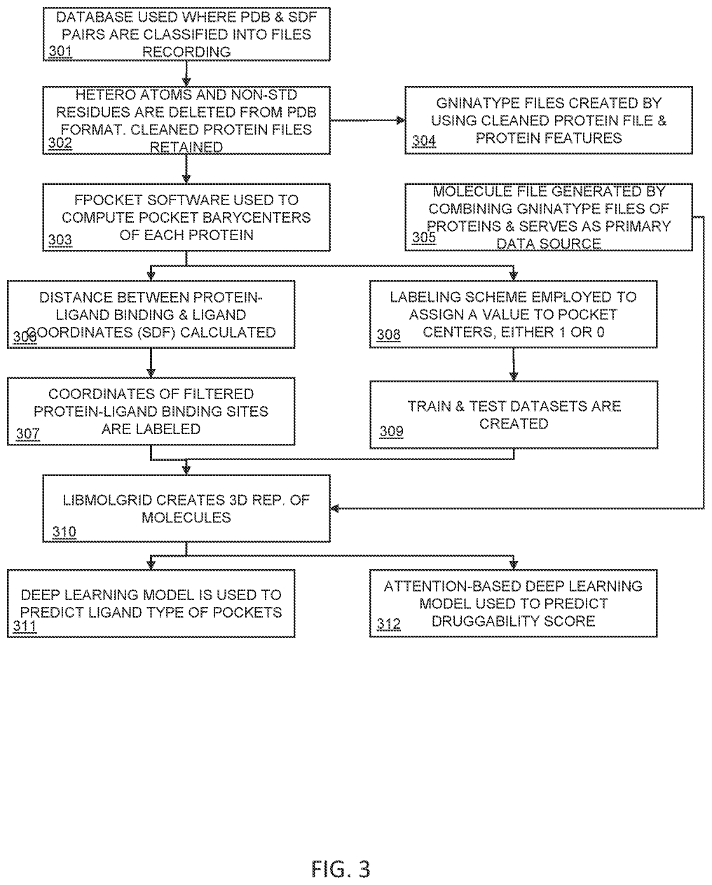

The embodiments the name of the invention is Druggability Score-Based Ranking and Ligand Type Classification of Protein-Ligand Binding Sites (RCLigand). The name of the invention itself gives away the fact that it consists of two primary components. As seen in FIG. 2 step 205, the prediction of ligand type and druggability score for protein-ligand binding sites requires the use of two distinct trained models. This means that there are two separate datasets and two separate training processes to perform these two main tasks. FIG. 2 illustrates the step-by-step process of the RCLigand software, from receiving the PDB input to producing the final result, Table 1. FIG. 3 shows the process of obtaining the trained models employed in FIG. 2 step 205. The preparation of the dataset used for druggability score prediction is shown in FIG. 3 step 308. FIG. 1 showcases the dataset employed for training the model designed to predict the ligand type of protein-ligand binding sites. FIG. 1, FIG. 2, FIG. 3, and Table 1 are explained step by step below in detail.

As shown in FIG. 1, step 101, the dataset preparation process starts with a public protein dataset such as PDBbind, scPDB, and so forth. In step 102, the CIF extension files of all proteins in the raw data set are downloaded and saved. CIF files are used for proteins because they contain more information about the protein than other file formats. In step 103, after the information extraction process, the proteins are categorized based on their ligand classes. This step can be described as a Natural Language Processing (NLP) problem because it involves extracting information from each protein's CIF file to determine the specific ligand type it is more likely to bind to. Five different types of ligands have been identified, namely antagonist, agonist, activator, inhibitor, and others. Then proteins that have been labeled in accordance with the kinds of ligands are kept in separate files inside a single dataset with all relevant file extensions. Step 104 is the update part of the data preparation process. When the dataset is wanted to be updated with new data, only the update module is run, and the missing data is added without running all operations again. During step 105, the protein-ligand binding sites of each protein are identified utilizing Fpocket. The barycenters of protein-ligand binding sites are determined in the x, y, and z coordinate planes. The distance between the barycenters and the ligand coordinates is measured to determine the pocket to which the ligand is bound. Ligand coordinate information is taken from the SDF file. In step 106, filtered protein-ligand binding sites are labeled according to the information obtained in step 103. As a result, a dataset containing protein-ligand binding sites labeled according to ligand types is prepared. If a protein-ligand binding site tends to bind to more than one type of ligand, this region is not added to the dataset in order not to mislead the training process.

In FIG. 2, step 201 displays the start of the flowchart where the PDB input is received from the end user, initiating the cleanup process. Clean PDB files are created by deleting all other non-standard residues except atom headers in the content of protein PDB files. This allows us to deal only with the atoms that make up the protein. In step 203, the gninatypes extension files for each protein are created using the libmolgrid library from the cleaned PDB files. Molgrid is a library that can be used for many different things. It uses multidimensional arrays of molecular data to show molecules in three dimensions. The file with the extension gninatypes contains the x, y, and z coordinate information and atom types of proteins. There are some protein features added when creating the file with the gninatypes extension, such as phosphorus, chlorine, bromine, etc. Different protein properties can be added as desired. In step 202, open-source software package Fpocket is one of the pre-existing geometry-based methods and an important part of our final deep learning architecture developed in the invention. Each protein has a different number of pocket regions. The barycenters of the pocket regions of each protein are listed in the x, y, z coordinate plane by using Fpocket. Step 204 is the input tensor data preparation part for our deep learning model. It is also called voxelization process. Libmolgrid has capabilities for sampling batches of data that are suitable for machine learning procedures. The idea of representing pocket coordinates as a multidimensional space by adding protein properties and using this as input in convolutional or recurrent neural networks was inspired by the Protein-Ligand Scoring with Convolutional Neural Networks research. There are other similar studies such as DeepPocket, RefinePocket, RecurPocket. Following the voxelization process, the problem becomes more aligned with the field of computer vision. Step 205 in FIG. 2 contains two separate train models obtained from FIG. 3. One of the trained models is for ligand type prediction of protein-ligand binding sites, and the other is for druggability score prediction. Training processes are explained in detail in FIG. 3. In step 207 there is an attention-based deep learning model to predict the ligand type. The attention module performs calculations for both channel-based attention and spatial-based attention on the tensor with dimensions of channel, height, width, and length. Since the tensor representing the protein after the voxelization process is 4D in size, it is important to weight each channel separately and apply spatial-based weighting to each tensor layer. The attention mechanism of the model is designed to give a more accurate result by using the information in the classification part of our invention. This deep learning model we used in our invention is another factor that makes our study unique. Likewise, step 206 is an attention-based deep learning model used to predict druggability scores of protein-ligand binding sites. In step 206, a modified version of the attention-based deep learning model is utilized, which differs slightly from step 207. The model here makes an estimation, also paying attention to the coordinates obtained in FIG. 1 step 106. In step 208, both the ligand type prediction and the druggability score prediction of protein-ligand binding sites are combined to form Table 1.

FIG. 3 presents a comprehensive flowchart illustrating the creation of two independently trained models utilized in step 205 of FIG. 2. Step 301 utilizes the database consisting of PDB and SDF pairs, which are saved under different files based on ligand types, as the input data. This dataset is obtained from step 103 of FIG. 1. Step 302 is the cleaning process of all Protein Data Bank (PDB) files. Hetero atoms and non-standard residues are deleted from the contents of the files in Protein Data Bank (PDB) format. In step 303, the protein-ligand binding sites of all the cleaned PDBs are identified using Fpocket. In step 304, gninatype files are created by utilizing the cleaned protein file and the protein features. The gninatype file is a file format that includes protein features. This file serves as a structured data format that encompasses essential information about the protein, enabling further analysis and processing in various computational tasks. In step 305, a molecule file is generated by combining all the gninatype files of proteins. This resulting molecule file, stored as a single file, serves as the primary data source for converting each coordinate into grid shape in the subsequent steps. Thanks to the gnina open-source program, the idea of creating a single molecule file using the information in the training and test datasets has emerged. This molecule file will be used later in step 310. In step 306, the protein's pocket barycenter coordinate points are compared to the protein's ligand coordinate points in terms of distance. This allows us to filter out the pocket coordinates to which the ligands bind. In step 307, the obtained filtered coordinate information is labeled according to the ligand types, and training and test datasets are created. Protein-ligand binding sites in training and test datasets are labeled according to five different ligand types. Those are antagonist, agonist, activator, inhibitor, and others. More ligand types can be added with minor updates in FIG. 1. Step 308 is the stage of preparing the train and test datasets that will be used for the training of the model developed for druggability score prediction. The dataset prepared for druggability score estimation consists of two distinct labels. The label “1” indicates that the binding site is considered druggable, while the label “0” indicates that the binding site is deemed non-druggable. In step 309, train and test datasets are created by taking a certain amount from each label to prevent unbalance situation. In step 310, libmolgrid creates a 3D representation of molecules by using molecule file. The generated grid is populated with train and test datasets. The depth of the tensor changes according to the number of protein properties we have determined in advance. In step 311, the training process is performed to predict the ligand type of pockets. Data augmentation is performed by rotating the coordinates to increase the variability of the training data. In step 312, the attention-based deep learning model for predicting the druggability score is trained using the training dataset obtained from step 309. Two separate trained tar files from both the prediction of ligand types and prediction of druggability score sections are saved and used in FIG. 2, step 205. Finally, the pocket sequence column in Table 1 is extracted from the CIF file of the relevant protein by using the coordinate information.

1. A method for a druggability score-based ranking and ligand type classification of protein-ligand binding sites (RCLigand), comprising:
creating a first training set for classifying ligand binding sites of proteins according to ligand types, wherein the first training set includes different types of ligand labels and their known binding sites on a target molecule;
creating a second training set for calculating a druggability score of protein-binding sites, wherein the second training set includes pocket and non-pocket coordinates;
identifying the position of the pocket coordinates associated with the binding of a known ligand type and assigning a corresponding ligand label to one or more regions defined;
detection of protein-ligand binding sites that tend to bind with more than one ligand type and not be included in the training process;
analyzing the regions for a first deep learning model in a calculation of a pocket druggability score, wherein said analyzing provides enhanced druggability score accuracy;
performing a deep learning process to a second deep learning model to classify one or more pockets, wherein the second deep learning model is configured to predict one or more ligand binding sites based on the ligand type; and
providing the predicted ligand type associated with the binding sites and predicting the druggability score of these regions as an output.

2. The method of claim 1, wherein creating the first and second training sets involves extracting ligand type information from protein files of formats including, but not limited to, Crystallographic Information File (CIF) and Protein Data Bank (PDB); and
further including a step of updating the first and second training sets with new data on ligand binding sites and their associated ligand labels, and where
data augmentation is performed by rotating the coordinates to increase the variability of the training and test data.

3. The method of claim 1, wherein the first deep learning model is an attention-based deep learning model to predict the druggability score, and wherein
the first deep learning model places additional attention on the coordinates of ligand binding sites during a training phase, and wherein
a permutation-based technique is utilized to determine the importance of each feature, thereby guiding the first deep learning model to emphasize additional features for more accurate predictions of druggability scores, and wherein
the prediction of druggability scores for each pocket is performed for any given input in a Protein Data Bank (PDB) format.

4. The method of claim 1, wherein the second deep learning model is an attention-based deep learning model to predict ligand type, and wherein
the development of channel and spatial-based attention mechanisms for multidimensional tensors in a deep learning model, and wherein
the model filters out ligand sites with a value below a particular druggability score, and wherein the model modifies a loss function of the second deep learning model to weigh with an increased emphasis to druggable sites, and wherein
the prediction of ligand types associated with each ligand binding site is conducted for any given input in a Protein Data Bank (PDB) format.

5. The method of claim 4, wherein upon providing the binding sites of the protein according to ligand types, a specific ligand type is selected, thereby enabling filtering of binding sites.

6. A computer device comprising a memory and a processor, wherein the memory stores a computer program and is characterized in that when executing the computer program, the processor implements steps of a method comprising:
obtaining a ligand type labeled training set, wherein
the training set includes protein-ligand binding sites' cartesian coordinates and atom types, and wherein
a first deep learning model is trained, and utilizes a set of weighted parameters that enable the first deep learning model to make predictions;
obtaining the training set being annotated with labels distinguishing between pocket and non-pocket regions, wherein
the training set includes protein-ligand binding sites' cartesian coordinates and atom types, and wherein
a second deep learning model is trained, and utilizes a set of weighted parameters that enable the second deep learning model to make predictions;
a molecular file is created with coordinate and type data, and a file is created by including different protein attributes without number limit for each protein;
a Protein Data Bank (PDB) file is received as an input, then processed by preserving atom-related headers while removing all other non-essential header information; and
utilizing the trained model to create a druggability score prediction for each ligand binding site.

7. The computing device of claim 6, wherein the second deep learning model is an attention-based deep learning model to predict ligand type, and wherein
the model filters out ligand sites with a value below a particular druggability score, and wherein
the model has both channel and spatial-based attention mechanisms for the protein tensor input, and wherein
the prediction of ligand types associated with each ligand binding site is conducted for any given input in a Protein Data Bank (PDB) format.

8. The computing device of claim 6, wherein the first deep learning model is an attention-based deep learning model to predict the druggability score, and wherein
the first deep learning model places additional attention on the coordinates of ligand binding sites during a training phase, and wherein
a permutation-based technique is utilized to determine the importance of each feature, thereby guiding the first deep learning model to emphasize additional features for more accurate predictions of druggability scores, and wherein
the prediction of druggability scores for each pocket is performed for any given input in a Protein Data Bank (PDB) format.
